(19) United States
(12) Patent Application Publication
(10) Pub. No.: US 2022/0253056 A1
(43) Pub. Date: Aug. 11, 2022

(54) Targeted Surface Sanitizing Robot

(71) Applicants: Kunal Ranjan, Cincinnati, OH (US); Krish Ranjan, Cincinnati, OH (US); Arjun Verma, Cincinnati, OH (US); Euan Ji, Cincinnati, OH (US); Nathan Padhy, Cincinnati, OH (US); Jack Brunner, Cincinnati, OH (US)

(72) Inventors: Kunal Ranjan, Cincinnati, OH (US); Krish Ranjan, Cincinnati, OH (US); Arjun Verma, Cincinnati, OH (US); Euan Ji, Cincinnati, OH (US); Nathan Padhy, Cincinnati, OH (US); Jack Brunner, Cincinnati, OH (US)

(21) Appl. No.: 17/169,518

(22) Filed: Feb. 7, 2021

(51) Int. Cl. G05D 1/00; G05D 1/02; A47L 11/40; A61L 2/24; A61L 2/10

(52) U.S. Cl. CPC G05D 1/0088; G05D 1/0212; G05D 1/0094; A47L 11/405; B25J 9/162; A47L 11/4011; A61L 2/24; A61L 2/10; A47L 11/4061

(57) ABSTRACT

A targeted surface sanitizing robot includes a transport drive and control system powered by batteries and arranged for autonomous movement of the robot to reach areas that need sanitizing. The robot chassis carries two arms that aid in sanitizing. The first arm has UV lights mounted on its underside. This arm swings horizontally and adjusts vertically to reach the surface to be sanitized. The second arm has a gripper at the end that can open a drawer or a closet so that the first arm can then sanitize that surface. The control system mounted on the robot is wirelessly connected to a remote central system that tracks which rooms or surfaces have been cleaned.

### INVENTION BACKGROUND

The present invention relates to the use of UV light to sanitize or disinfect specific horizontal surfaces in a room without letting the UV light fall on other surfaces/objects/living entities in the room that may potentially get damaged by the UV light (e.g. plants or human exposure).

UV light is commonly used to disinfect rooms. Several companies have launched room sanitizing units in the market (UVD Robots and Xenex). Examples of such devices are the Tru-D, Surfacide™, and Artz (Automatically Reliably Targeting Zero) by American Ultraviolet Co., Inc. of Lebanon, Ind. Each of these devices uses UV-C lamps to accomplish the sanitization task. Timers and sensors to prevent human exposure to UV and to determine that the right dose of UV has been applied. However, there is one feature missing in these UV based room sanitizing devices. The UV light is spread in the room using a vertically mounted tower. When this tower-shaped UV light lights up, it spreads UV light in the entire room. This necessitates that any humans who are in the area need to exit the room being sanitized. Also, the UV light can affect the plants or other UV sensitive material in the room. In addition, the UV light is not concentrated, instead, it is dispersed, resulting in lower accuracy of sanitization. This problem needs to be solved.

### BRIEF SUMMARY OF INVENTION

The present invention addresses the problem by developing a system that disinfects specific horizontal surfaces instead of disinfecting the entire room. This device is mounted on a robotic platform which may be based on an appliance such as the iRobot Create®, marketed by the iRobot company of Bedford, Mass., to move through the room while avoiding obstacles. The platform may also be based on similar systems that are currently available. Examples of these systems include the iRobot Roomba®, iRobot Ava®, and the Bilibot. The Roomba® is a commercially available automated vacuum cleaner, the Ava® is a commercially available mobile robot platform and the Bilibot is an open-source robotic platform for hobbyists that combines the iRobot Create® platform, Microsoft® Kinect®, and a robotic arm. At least certain of these systems, such as the Ava®, are capable of holding up to 150 lbs, without affecting mobility. Navigation is done using different types of sensors like cameras, ultrasonic distance sensors, light beams, or a guiding line with the color marking on the floor. These allow the invention to reach specific horizontal areas to be sanitized.

This invention builds on the previously patented robotic room sanitizers (for example Patent No. WO2014039076A1) and adds specific features to make it more specific to be used in a typical setting in a commercial, school, or medical facility.

The appliance according to the present invention may include: i) several high output UV lamps that are mounted on a swing arm that can move in the x-direction or raised vertically to adjust to the height of the horizontal area to be sanitized while navigating the room. (ii) a second arm that has a gripper at the end that can open a drawer or closet so that the first arm can sanitize the horizontal surface inside (iii) an AC inlet to a DC battery, which in turn powers the lamps, sensors, and processors 3) a system via the processor to automatically shut off the lamps, when the device is moving between surfaces to be sanitized. 5) a wireless connection to a centralized remote location to emit a signal indicating that a certain room has been treated, and 6) a robotic platform with a navigation system that will carry the above components.

Such a system will reduce the energy needed by the lamps because the distance between the lamps and the surfaces to be sanitized will be reduced. The number of lamps needed will also be reduced. Thus the cost of the unit and the cost of ownership will be lower.

### BRIEF DESCRIPTION OF DRAWINGS

FIG. 1 is a block diagram of the electrical and control system according to the present invention.

FIG. 2 is a pictorial perspective view showing the structure of an exemplary embodiment of the system according to the present invention.

### DETAILED DESCRIPTION OF INVENTION

The invention provides a device to target specific horizontal areas to disinfect using UV light. The device consists of a main structural housing on which two kinds of arms are mounted. The first is a two-axis arm. It moves in the x-y direction to reach the surface to be cleaned. Different rooms may have different heights of surfaces to be sanitized, so this arm can also move vertically up and down to adjust itself to the height of the surface to be sanitized. The second arm is a robotic arm with a gripper. This gripper can open the drawers or doors of closets. This second arm works in conjunction with the first arm. The second arm opens the doors/drawers and then the first arm sanitizes that surface. The main structural housing consists of a chassis on omnidirectional wheels that can be guided in any direction based on the path to the surface to be sanitized.

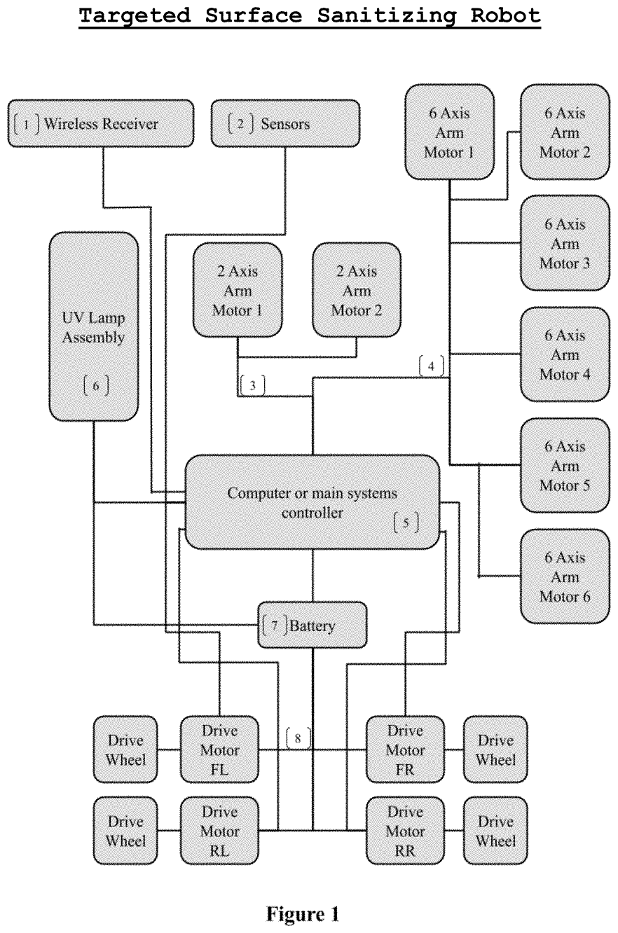

The main structural housing also houses all the batteries, electronics, sensors, and motors. The appliance according to the present invention shown in FIG. 1 includes a computer or microprocessor (5) containing, in a conventional manner, memories storing firmware and software for controlling the movement of the appliance and operation of high output UV lamp assemblies (6). Computer (5) can be programmed to automatically shut off the lamps and move the device to the next location to be sanitized when the prescribed dose of UV has been delivered. The appliance further includes a battery (7) that supplies power to the computer (5) and to the UV lamps, as well as other components to be described below. Operating power is supplied from a battery (7) to the lamps of assemblies (6) via a DC connection. The appliance further includes four drive motors (8), each coupled to rotate a respective drive wheel. Drive motors (8) are powered from a battery (7) and controlled by signals from a computer (5). The appliance will typically be provided with at least one further wheel, which need not be driven or steerable, but which can be supported to pivot freely about a vertical axis. Steering is accomplished by separately controlling the rotation of each of the wheels. The appliance also includes a wireless transceiver (1), connected to a computer (5), for wireless communication with a centralized remote location to emit signals that can identify the location of the appliance and indicate that a certain room has been treated. The appliance further includes sensors (2) connected to a computer (5) for guiding and controlling the movement of the appliance according to known techniques. These sensors may be of the types described earlier herein.

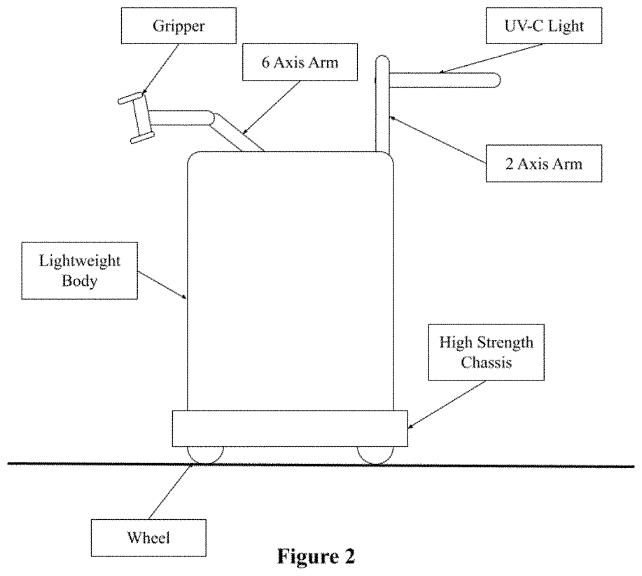

FIG. 2 is a pictorial view showing the structure of an exemplary robotic appliance according to the invention. This appliance is supported on at least three wheels, two of which are wheels 12 of FIG. 1, and carries all the other components shown in FIG. 1. The appliance includes a robotic platform that will carry the above components and allow them to be moved freely throughout a room.

The robotic platform has two arms. The first arm is an arm that can swing to the horizontal surface to be sanitized. This arm can also move vertically up and down to adjust to the height of the surface to be sanitized. There are UV-C lamps mounted on this arm and these lamps disinfect the surface when lit. The UV lamps turn on only when the arm swings to the surface to be sanitized. This conserves battery power.

The second arm is a 6-axis arm (4) that has a gripper at the end. This arm works in conjunction with the previous arm. This arm will grip and open the drawers so that the UV light can sanitize whatever is inside the drawer.

The robot has an onboard system that stores the configuration of each room to be sanitized. As part of the setup, the robot needs to be taught the areas to be sanitized in each room including the path to follow. The robot accesses that information as it goes to each room. As the cleaning robot moves to the different locations within the room, it sanitizes as per the configuration stored for that room. This includes either just using the first arm to swing on top of the horizontal surface to be cleaned or using a combination of swing arm and gripper arm to do that.

What is claimed:

1. A robotic appliance for sanitizing horizontal surfaces in a targeted manner so as not to cause adverse effect to other objects in the room, comprising:
an autonomous mobile platform equipped to travel over a floor to cover the objects in the room to be sanitized; two swing arms with one arm with UV light on the under-surface—this arm swings horizontally on the surface to be sanitized as well as moves up and down to adjust to the height of the surface to be sanitized and a second swing arm with a gripper to open drawers or other cabinets so that the contents inside the drawers can also be sanitized.
The above platform will carry a rechargeable battery power source to move the platform, the arms, and power the UV light. It will also consist of a control system that will navigate the robot to the designated location, swing the arm, and/or use the gripper to move obstacles/open compartments for the duration needed to sanitize the surface.
